(19) United States
(12) Patent Application Publication
(10) Pub. No.: US 2025/0205514 A1
(43) Pub. Date: Jun. 26, 2025

(54) SYSTEMS AND METHODS OF DETERMINING TISSUE PROPERTIES FOR CT-BASED RADIATION THERAPY PLANNING

(71) Applicant: Washington University, St. Louis, MO (US)

(72) Inventors: Jeffrey WILLIAMSON, St. Louis, MO (US); Maria MEDRANO, St. Louis, MO (US); Xinyuan CHEN, St. Louis, MO (US); Tianyu ZHAO, St. Louis, MO (US); Joseph O'SULLIVAN, St. Louis, MO (US); Lucas Norberto BURIGO, St. Louis, MO (US)

(21) Appl. No.: 18/844,610

(22) PCT Filed: May 11, 2023
(86) PCT No.: PCT/US2023/021901

§ 371 Date: Sep. 6, 2024

Related U.S. Application Data
(60) Provisional application No. 63/340,800, filed on May 11, 2022.

(51) Int. Cl. A61N 5/10; G06N 20/00; G16H 20/40; G16H 30/40; G16H 50/50

(52) U.S. Cl. CPC A61N 5/103; G06N 20/00; G16H 20/40; G16H 30/40; G16H 50/50; A61N 2005/1087

(57) ABSTRACT

A method of predicting planning parameters for proton radiation including receiving training data of tissues and executing a model. The model is trained by fitting the model with training data and adjusting model parameters during fitting. The model is configured to predict planning parameters required in a proton radiation planning system in generating a proton radiation plan of a subject.

### CROSS REFERENCE TO RELATED APPLICATIONS

This application claims the benefit of and priority to U.S. Provisional Patent Application No. 63/340,800, filed May 11, 2022, entitled “JOINT STATISTICAL DUAL-ENERGY CT IMAGE RECONSTRUCTION,” which is hereby incorporated by reference in its entirety.

### STATEMENT REGARDING FEDERALLY SPONSORED RESEARCH & DEVELOPMENT

This invention was made with government support under CA212638 awarded by the National Institutes of Health. The government has certain rights in the invention

### BACKGROUND

The field of the disclosure relates to radiation planning, and more particularly, to systems and methods of radiation planning based on CT imaging.

Proton-beam therapy is safer and as effective as traditional therapy for cancer because of the high gradient dose fall-off near the end of the proton range, which improves dose conformity while sparing normal tissue from radiation. Computed tomography (CT) data are used in dose calculation and radiation planning. Additional 2-3.5% of safety margins of the proton range are, however, included to count for uncertainty in current dose calculation methods based on CT. Therefore, improvements in accuracy of patient specific radiation planning are desired.

This background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### DRAWINGS

FIG. 1 is a flow diagram of an example process for predicting planning parameters, according to at least one embodiment of the present disclosure.

FIG. 2 is a figure illustrating an example proton beam dose deposition vs. tissue depth, where the solid black line illustrates the spread-out Bragg Peak (SOBP) region in proton radiotherapy.

FIG. 3 is a schematic illustrating an example Joint Statistical Image Reconstruction method with a Basis Vector Model (JSIR-BVM) from reconstructing basis vector model weights.

FIG. 4 is a schematic flow chart illustrating an example TOPAS Monte Carlo simulation method

FIG. 5 is an example 3-D plot showing parameterization of soft tissue.

FIG. 6 is an example 3-D plot showing parameterization of bony tissue.

FIG. 7 is example values for percent root mean square error (RMSE) in atomic fraction of 70 tissues for BVM and SECT

FIG. 8 is an example of Predicted composition (%) vs. benchmark composition (%) for carbon and oxygen for BVM indexing.

FIG. 9 is an example of Predicted composition (%) vs. benchmark composition (%) for carbon and oxygen for SECT indexing.

FIG. 10 is example Percent Error (%) for Stopping Power Ratio (SPR) Error for SECT Material Indexing and BVM Material Indexing.

FIG. 11 is example R80 Percent Error (%) for single-energy CT (SECT) Material Indexing and BVM Material Indexing.

FIG. 12 is an example of the integrated depth dose profile of proton beam delivered to whole vertebral column and cartilage tissue with ground truth, BVM, and SECT material indexing compositions in Dose (%) vs. Tissue depth (cm).

FIG. 13 is an example of lateral profile of proton beam delivered to Yellow Marrow tissue with ground truth, BVM, and SECT material indexing compositions in Dose (%) vs. Lateral position (cm).

FIG. 14 is example scatter plots showing linear relationship between percentage composition values for hydrogen, carbon, nitrogen, oxygen, phosphorus, and calcium and scaled c′₁, c′₂, and r′_{c} BVM parameters for (a) soft and (b) bony tissues r′_{c} intervals. Elemental weights in c′₁/c′₂/r′_{c} parametrization is displayed in c′₁/c′₂ space for illustration purposes.

FIG. 15 is an example showing resulting fits of BVM material indexing parametrization applied on benchmark fractional elemental composition of reference soft and bony tissues as well as corresponding soft and bony tissue root-mean-square (RMS) and maximum percentage point error between predicted elemental composition and their respective benchmark values for the two investigated methods.

FIG. 16 is an example plot showing overall discrepancies between predicted and benchmark ρₘ (mass density), I-value (mean excitation energy), and SP (stopping power) tissue parameters for BVM ((a), (c), and (e)) and HU ((b), (d), (f)) material indexing methods. The x-axis denotes each of the 46 and 24 soft and bony tissues in the reference material list. The black dotted line represents perfect correspondence, and the red dotted lines enclose the −1% to 1% error interval.

FIG. 17 is an example plot showing overall discrepancy between R80 (depth of 80% dose) corresponding to BVM material indexing method and GT (ground truth). The x-axis denotes each of the 46 and 24 soft and bony tissues in the reference material list. Black dashed line represents perfect correspondence, and the red dashed lines enclose-1 to 1 mm difference interval.

FIG. 18 is an example plot showing overall discrepancy between R80 corresponding to HU material indexing method and GT. The x-axis denotes each of the 46 and 24 soft and bony tissues in the reference material list. Black dashed line represents perfect correspondence, and the red dashed lines enclose-1 to 1 mm difference interval.

FIG. 19 is an example plot showing representative scored IDD (integrated depth-dose) curves for soft tissues ((a) and (b)) and bony tissue with highest (c) R80 discrepancy between HU and BVM material indexing methods.

FIG. 20 is an example plot showing representative lateral dose curves for soft tissues ((a) and (b)) and bony tissue (c) with highest FWHM80 discrepancy between HU and BVM material indexing methods.

FIG. 21 is an example scatter plot showing deviations of ground truth % composition values (symbols) of H, P, and Ca from BVM material indexing predictions (lines) in bony tissue as functions of c₁ and c₂.

FIG. 22 is an example plot showing integrated depth dose (IDD) (zoomed to the Bragg peak dose range) of brain cerebrospinal fluid showing the ground truth and SECT and BVM predictions.

FIG. 23 is an example plot showing errors in depth of 80% of the maximum IDD from Monte Carlo simulations using material files derived from BVM indexing for 69 reference tissues.

FIG. 24 is an example plot showing errors in depth of 80% of the maximum IDD from Monte Carlo simulations using material files derived from SECT indexing for 69 reference tissues.

FIG. 25 is a block diagram of an example computing device.

### BRIEF DESCRIPTION

In one aspect, a computer-implemented method of predicting planning parameters for Monte Carlo proton radiation planning on a subject, is provided. The computer implemented method includes receiving training data of tissues, wherein for each tissue, the training data includes elemental compositions of a plurality of elements in the tissue, a mass density of the tissue, and basis material weights when a linear attenuation coefficient of the tissue is represented as a weighted sum of linear attenuation coefficients of basis materials and executing a linear regression model, wherein an elemental composition includes a mass fraction of one of the plurality of elements in a tissue, and the model includes that the mass fraction is a linear function of the base material weights. Training the model includes fitting the training data with the model and adjusting model parameters of the model during the fitting. The trained model is configured to predict planning parameters required in a Monte Carlo proton radiation planning system in generating a proton radiation plan of a subject. The planning parameters include elemental compositions and mass densities of tissues in a treatment region of the subject.

In another aspect, a computer-implemented method of predicting planning parameters for Monte Carlo proton radiation planning on a subject is provided. The method includes receiving raw data of a subject acquired using a computed tomography system and deriving basis material weights at each image voxel location based on the raw data, wherein the basis material weights are weights in expressing a linear attenuation coefficient of a tissue at the image voxel location as a weighted sum of linear attenuation coefficients of basis materials. The method includes executing a linear regression model and estimating planning parameters including elemental compositions of a plurality of elements and/or a mass density of the tissue at the image voxel location based on the basis material weights using the model. The method includes outputting the planning parameters. The planning parameters are required in a Monte Carlo proton radiation planning system in generating a proton radiation plan of the subject.

In yet another aspect, a computer-implemented method of predicting planning parameters for Monte Carlo proton radiation planning. The computer implemented method includes receiving raw data of a subject acquired using a computed tomography system and deriving basis material weights of a plurality of basis materials at an image voxel location based on the raw data and executing a machine learning model. The method further includes estimating planning parameters including elemental compositions of a plurality of elements and/or a mass density of tissues in the image voxel location using the model based on the basis material weights and outputting the planning parameters. The planning parameters are required in a Monte Carlo proton radiation planning system in generating a proton radiation plan of the subject.

### DETAILED DESCRIPTION

The disclosure includes systems and methods of identifying planning parameters such as mass density and elemental composition for more accurate Monte Carlo-based dose calculation in proton-beam radiotherapy based on CT data. The Monte Carlo-based dose calculation system is described herein as an example for illustration purposes only. Systems and methods may be applied to other radiation planning systems.

Systems and methods described herein provide predictions of planning parameters required by the Monte Carlo proton radiation planning system. The required planning parameters include independent variables of elemental compositions of six elements and mass density for tissues in each voxel of the imaging region, based on two related basis material weights c₁ and c₂. Deriving seven independent variables from two related variables is mathematically challenging. Systems and methods described herein are advantageous in provide a solution to this challenging problem and increasing accuracy in predicting planning parameters, thereby realizing the full potential of proton radiation therapy as a safe and effective modality compared to traditional radiation therapy. Further, in known systems and methods, low mass density tissues such as inflated lung are typically not included in the prediction. Systems and methods described herein are advantageous in including tissues that are not typically included in known models, thereby increasing the applicable tissues and organs for radiation planning.

Previous studies demonstrate that a linear basis-vector model (BVM) predicts proton stopping-power ratio (SPR) maps in simulated and experimental data. While SPR may be used for pencil-beam dose calculations, at least some known proton radiation planning systems, such as Monte Carlo (MC) simulations, requires the atomic composition and density of each medium to compute multiple elastic, inelastic, and nuclear scattering cross-sections. Proton radiation therapy (PRT) has the potential to improve cancer treatment outcomes because of their unique physical characteristics: a dramatically rapid dose falls off to near zero at the high-energy proton range. By modulating the proton energy, one would align this dose-off with the distal margin of the clinical target volume (CTV), so that the normal tissues and organs would be spared from exposure to high doses, reducing treatment complications. In practice, the accuracy with which this depth of penetration (AKA proton range) can be predicted from standard single-energy CT (SECT) is limited to about +/−3.5%. To ensure that the CTV receives adequate dose, a safety margin of 2-8 mm must be added to the distal CTV boundary which greatly diminishes the normal tissue-sparing potential of PRT.

A Joint Statistical Image-Reconstruction (JSIR) process operates jointly on raw CT data (sinograms) acquired at two different scanning energies to create highly uniform, artifact-free quantitative images. JSIR is based on a Basis-Vector Model, which represents linear attenuation coefficients (LACs), a quantity closely related to CT image intensity, at any energy as a linear combination of LACs of two dissimilar basis materials. The output of JSIR are the two basis-weight images.

Dissimilar basis materials may include two materials having different compositions. Dissimilar basis materials may include more than two materials having different compositions, e.g., three materials having different compositions or four materials having different compositions. Different compositions may refer to different configurations, types, and/or ratios of atoms or molecules. For example, in some embodiments, the two dissimilar basis materials may include polystyrene and CaCl₂).

In another embodiment, a linear basis-vector model (BVM) is extended to create highly accurate images of proton stopping power (SP), SP ratios (SPR), electron densities, and I-values. The resultant process is called JSIR-BVM. An uncertainty analysis has demonstrated that JSIR-BVM can image SP and SPR in patients with a total uncertainty (coverage factor k=1) less than 1%. This results in sub-percentage uncertainties in range predicted by pencil-beam dose calculations, dramatically reducing the need for large range-uncertainty margins.

As used herein, a subject is a human, animal, or a phantom, or a part of a human, an animal, or a phantom, such as an organ or tissue. A vertebral column, cartilage, and yellow marrow are used as example subjects for illustration purposes only. Systems and methods described herein may be applied to other subjects, e.g., organs or tissues, composed of bony and/or soft tissues.

Embodiments disclosed herein addresses at least the following problem: Implementation of Monte Carlo (MC) simulation (one of the most accurate dose-calculation tools available) requires specification of more radiological quantities required than just SP, which quantifies continuous energy loss by protons. MC includes inelastic and elastic cross sections for protons and electrons, along with nuclear cross sections and neutron-transport cross-sections as functions of energy, all which depend on elemental composition of the tissue as well as density. Furthermore, pencil-beam dose calculations require additional, tissue composition-dependent quantities, e.g., mass scattering power and parameters for modeling the nuclear halo. Embodiments of a material-indexing provides a tool for predicting mass densities and atomic composition of human soft and bony tissues from the two BVM-weight images produced by our JSIR-BVM DECT image-reconstruction process. Embodiments of the method preserves sub-percentage uncertainty in range predicted by the dose calculation-algorithm as well as lateral pencil-beam profiles and nuclear halo distributions.

A database of 69 standardized elemental tissue-composition formulations was derived from the literature to train a linear regression model, referred to as Ground Truth (GT). The BVM weights, (c₁, c₂) were calculated for each tissue, along with the fractional concentrations by weight, [H], [C], [O], [N], [P], and [Ca]. For each these 6 elements Xi, the following model was fit to these data, separately for bony and soft tissues.

$${X}_{i} = {a}_{0. i} + {a}_{1. i} {c}_{1} + {a}_{2. i} {c}_{2} + {a}_{3. i} \frac{{c}_{1}}{{c}_{1} + {c}_{2}} \qquad ( 1 )$$

The atomic mixture rule was used to estimate the mass density given the electron density estimated via the BVM model. The resultant tissue compositions were used to define custom materials for the TOPAS freely-available proton-therapy Monte Carlo code using a parameter-file input system.

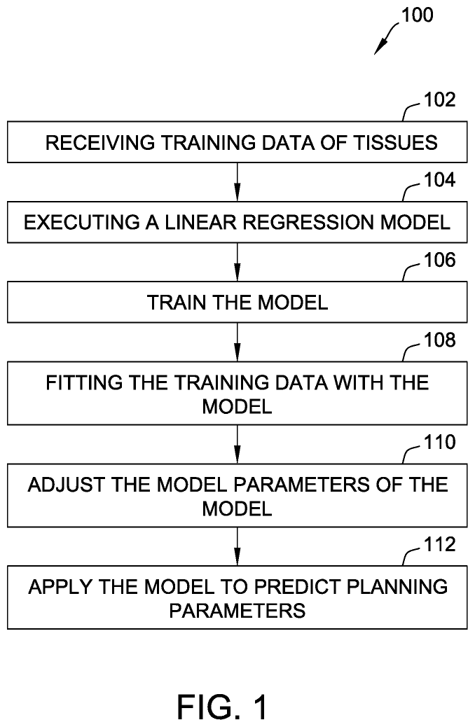

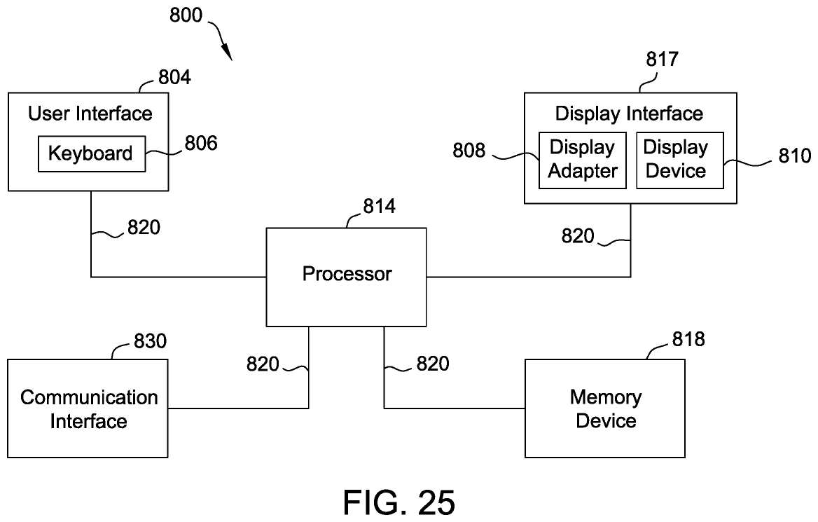

FIG. 1 illustrates a flowchart of an example process 100 for predicting planning parameters for Monte Carlo proton radiation planning on a subject. In the example embodiment, process 100 may be implemented by a computing device, e.g., computing device 800, shown in FIG. 25. In other embodiments, process 100 may be implemented using other or additional computing devices.

In the example embodiment, process 100 includes receiving 102, e.g., by a computing device, training data of tissues. The training data of tissues includes elemental compositions of a plurality of elements in each tissue. In some embodiments, the training data of tissues may include 70 different tissues. The training data of tissue may include a mass density of each tissue, basis material weights where a linear attenuation coefficient of the tissue represented as a weighted sum of linear attenuations coefficient of basis materials. In some embodiments, the process may include normalizing the basis material weights to account for a tissue having a low mass density into the model by scaling the basis material weights with a factor.

In the example embodiment, process 100 includes executing 104 a linear regression model. An elemental composition includes a mass fraction of one of the plurality of elements in a tissue. The model may include the mass fraction that is a linear function of the base material weights.

In the example embodiment, process 100 includes generating a model. The model may be the executed linear regression model. In other embodiments, the model may be any suitable machine learning model. The model may be generated by training 106 the model using the received training data, e.g., by fitting 108 the model to the training data. In some embodiments, the model may be generated by tuning the model, e.g., by adjusting 110 one or more model parameters to fit the model to the training data. The model may include that the mass fraction is a linear function of a weighted component ratio between the basis material weights. The weighted component ratio may be derived based on the basis material weights and electron densities of the basis materials. In some embodiments, the model includes that the mass fraction is a linear function of the normalized basis material weights and a weighted component ratio between the normalized basis material weights.

In some embodiments, training may include, for each tissue, estimating the mass density of the tissue based on electron densities of the basis materials and the basis material weights. In some embodiments, training may also include comparing the estimated mass density with the mass density of the tissue in the training data. In some embodiments, training may also include adjusting the model based on the comparison.

In some embodiments, the model may include a first submodel of a soft tissue and a second submodel of a bony tissue. Fitting the training data may include bracketing materials into the soft tissue and the bony tissue based on the BVM weight ratio and applying the first submodel or the second submodel on the training data based on the bracketing.

In the example embodiments, the model is associated with one or more model inputs and one or more model outputs. For example, application of one or more model input(s) to the model generates one or more model outputs. Model inputs may include c₁ and c₂ of a JSIR of one or more sinograms from a subject tissue. The subject includes tissue and/or tissues. Model inputs c₁ and c₂ may have been determined from images or scans of a target region including a tumor and surrounding tissues that are in proximity to the tumor. Target region proximity tissues may include tissues superficial to the tumor and/or tissues deeper than the tumor. Proximity tissues may also include any tissues in a region surrounding the tumor. In other embodiments, model inputs may include any suitable model inputs that may be applied to the model to generate model outputs. Process 100 may include applying 112 the model inputs to determine one or more model outputs.

In the example embodiment, model outputs may include an elemental composition of the tissue of the target region. Model outputs may include mass density of elemental composition of the tissue in the target region. Model outputs may include planning parameters required by for a radiation planning system, e.g., such as a Monte Carlo proton radiation system.

### EXAMPLES

### Example 1

A first example, regarding predicting Material Composition and Density from Basis-Vector Model Weights for Dual-Energy CT-Based Monte Carlo Proton-Beam Dose Calculations, is provided.

Previous studies demonstrate that a linear basis-vector model (BVM) accurately predicts proton stopping-power ratio (SPR) maps in simulated and experimental data. While SPR is sufficient for pencil-beam dose calculations, Monte Carlo (MC) simulation requires the atomic composition and density of each medium to compute multiple elastic, inelastic, and nuclear scattering cross-sections. Embodiments described herein includes methods for predicting atomic composition and mass density from the two independent BVM weights derived from dual-energy CT imaging.

In some embodiments, a BVM material indexing method, includes multiple linear regression on the BVM weights and their quotient to predict the percent by weight concentration of elements for Z=1:20 and mass density of 69 representative tissue-compositions derived from the literature. The predicted compositions and densities were imported to the TOPAS MC codes and used to simulate a single 200 MeV proton beam delivered to uniform cylinder phantoms composed of the 69 tissues. MC dose distributions based on the BVM and a standard single-energy CT (SECT) material indexing approaches were compared to those derived from ground-truth tissue atomic compositions. The SPR, range (RBP), and depth of 80% of maximum dose (R80) were utilized to quantify dose-estimation errors.

In some embodiments, the method may result in a root-mean-square (RMS)/Max error in estimated SPR and RBP were 0.6/2.1% and 1.3/5.2 mm for SECT and 0.1/0.3% and 0.3/0.6 mm for BVM material-indexing schemes. Similarly, RMS/Max R80 errors for bony (soft) tissues for the SECT and BVM approaches were 0.7/1.5 mm (1.6/5.3 mm) and 0.1/0.3 mm (0.3/0.7 mm), respectively. The method of two-parameter BVM space for material indexing dramatically improves TOPAS MC dose-calculation accuracy (by factors of 4 to 7 in RMS) compared to the standard SECT single-parameter indexing process.

### Example 2

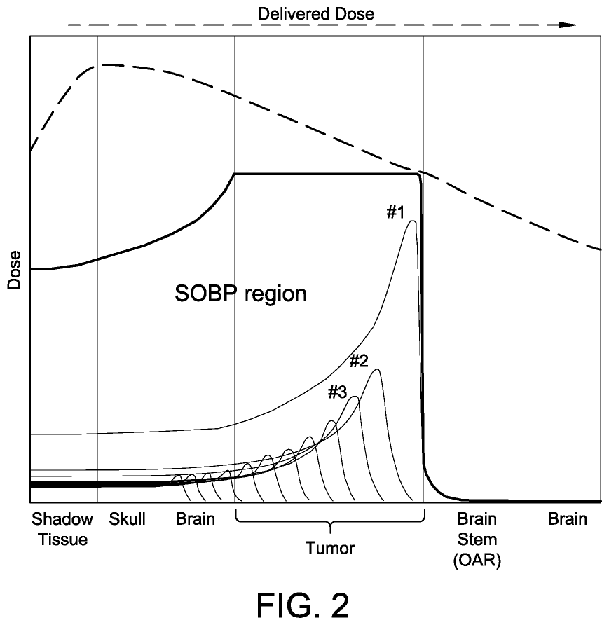

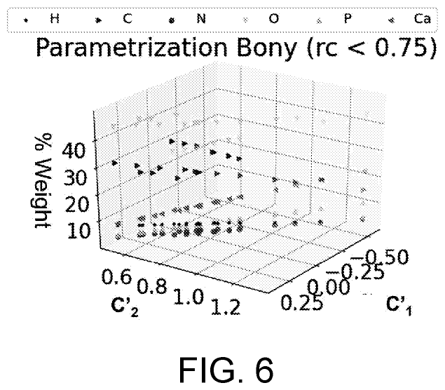

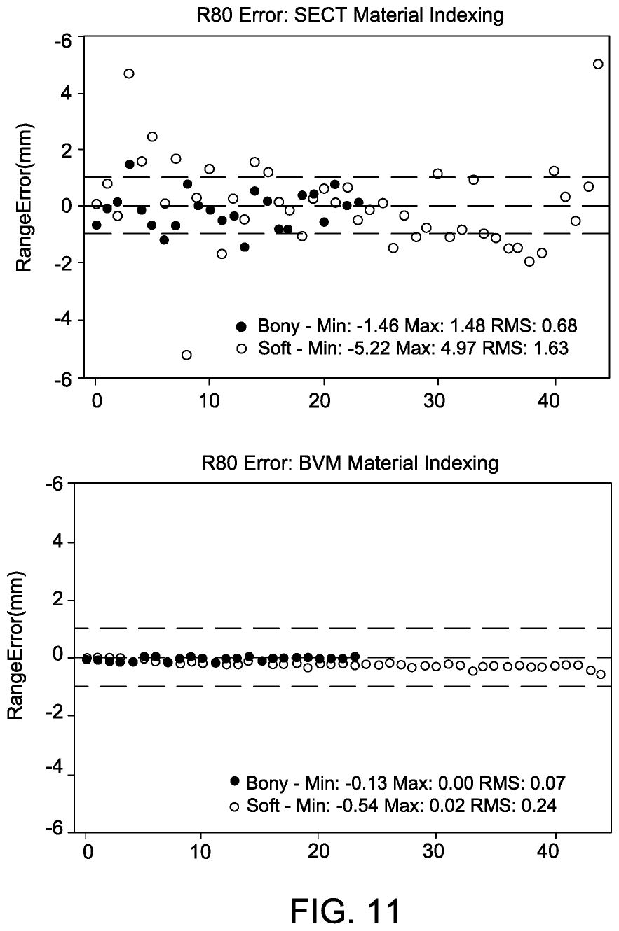

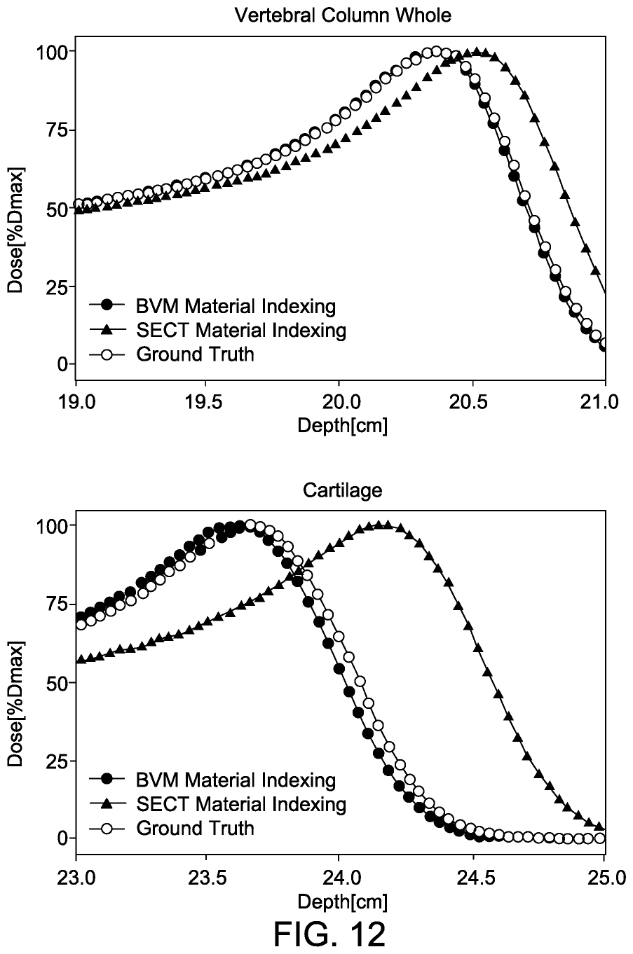

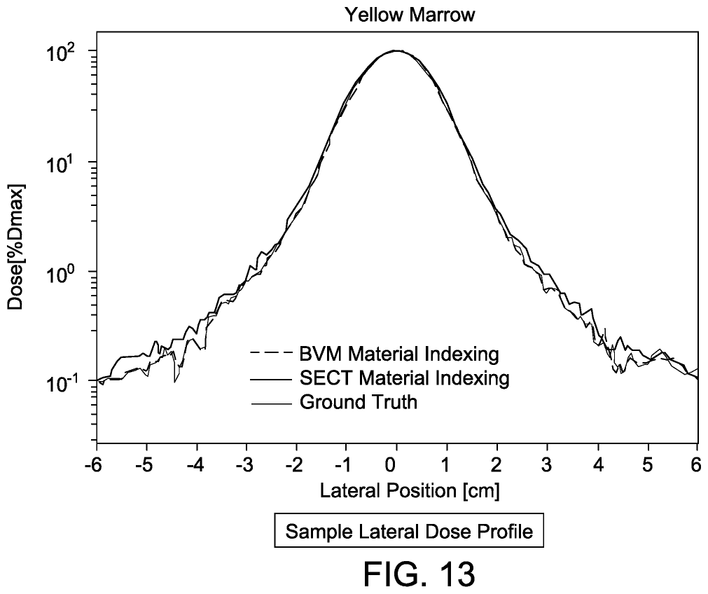

In reference to FIGS. 2-13, a second example, example 2 regarding Predicted Material Composition and Density from Basis Vector Model Weights for Dual-Energy CT-Based on Monte Carlo Proton Beam Calculations is provided.

The systems and methods described herein improve Proton Therapy for conformal tumor treatment and dose delivery. Range uncertainty is associated with the unknown depth of penetration of the proton beam, e.g., the depth of the proton beam past/deeper than the target region of the tumor. The systems and methods, described herein, accurately determines atomic tissue composition and density to more accurately determine the penetration depth of the proton beam. Range uncertainty may be caused by various sources of uncertainty, such as image streaks, residual cupping artifacts, noise, tissue composition variations, and signal formation model mismatch. In some embodiments described herein, tissue composition and density are determined using a Joint Statistical Image Reconstruction method with a Basis Vector Model (JSIR-BVM) which improved the accuracy of the range uncertainty of the proton beam depth, over conventionally used methods of determining concentration, such as standard single-energy CT (SECT). For comparison, SECT may have a range uncertainty of between 2.5%-3.4% (1-8 mm), while the system and methods described herein utilizing the JSIR-BVM may determine a range uncertainty between 0.6%-0.9% (1-2 mm).

In reference to FIG. 2-4, systems and methods described herein include iteratively reconstructing c₁ and c₂ basis vector model weights according to the function: μ(x,E)=c₁(x)μ₁(E)+c₂(x)μ₂(E). The c₁ and c₂ basis vector model weights are used to determine mass density (ρₘ) and elemental composition, such as H (Hydrogen), C (Carbon), O (Oxygen), Ca (Calcium), and/or P (Phosphorus). Images, e.g., CT-Images, may be obtained of a subject, using 90 kVp or 140 kVp, or both 90 kVp or 140 kVp voltage source. In some embodiments, sinograms may be collected of the subject.

The method may include deriving benchmarks for c₁′, c₂′, and r_{c} for 70 representative tissues form atomic compositions and CT scanning spectra.

$${c}_{i}^{′} = {c}_{i} \cdot \frac{{ρ}_{e , 1}}{{ρ}_{e , 2}} .$$

The method includes fitting the mass fraction of six main elements (H, C, N, O, Ca, and P) to benchmark c₁′, c₂′, and r_{c} by two sets of linear regression models, such that;

$${w}_{k , x} = {α}_{1 , k} {c}_{1 , y}^{′} + {α}_{2 , k} {c}_{2 , y}^{′} + {α}_{3 , k} {r}_{c , y}^{′} + {α}_{4 , k} , ( {r}_{c} \geq \text{.75} ) \qquad ( 1 )$$

$${w}_{k , x} = {β}_{1 , k} {c}_{1 , y}^{′} + {β}_{2 , k} {c}_{2 , y}^{′} + {β}_{3 , k} {r}_{c , y}^{′} + {β}_{4 , k} , ( {r}_{c} < . 75 ) \qquad ( 2 )$$

$${R}_{c} = \frac{{\hat{c}}_{1} {ρ}_{e , 1}}{{\hat{c}}_{1} {ρ}_{e , 1} + {\hat{c}}_{2} {ρ}_{e , 2}} \qquad ( 3 )$$

The method further includes calculating I-value using material composition and Bragg Additivity Rule. The method may further include applying an atomic mixture rule to an estimated electron density to determine mass density of the subject material.

$${ρ}_{e} = {c}_{1} {ρ}_{e , 1} + {c}_{2} {ρ}_{e , 2} . \qquad ( 4 )$$

A TOPAS Monte Carlo Simulation may include determining a material definition including SECT material indexing, BVM material indexing, and Ground truth composition. The TOPAS model may include supplying a proton beam to a subject, such as a solid cylinder test subject, to determine an integrated depth dose. The TOPAS Monte Carlo Simulations includes determining the range of depth for the dosage (%) using Bragg Peak Width.

Analytical and Monte Carlo Results: The BVM material indexing was compared to the conventional SECT material indexing method which used a HU/Material Composition look up table and HU/ρₘ linear relationship. The Monte Carlo simulations may be performed in TOPAS with a monoenergetic 200 MeV pencil beam consisting of 2×10⁵ particles in a Gaussian Distribution with σ=4 mm.

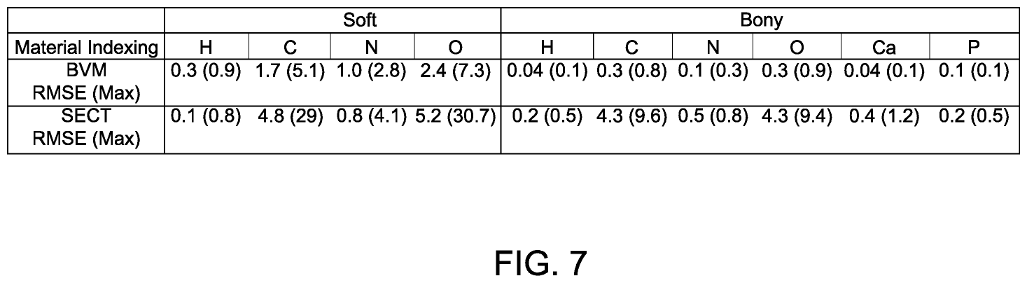

Percent Error in Atomic fraction for 70 tissues is shown in FIG. 7.

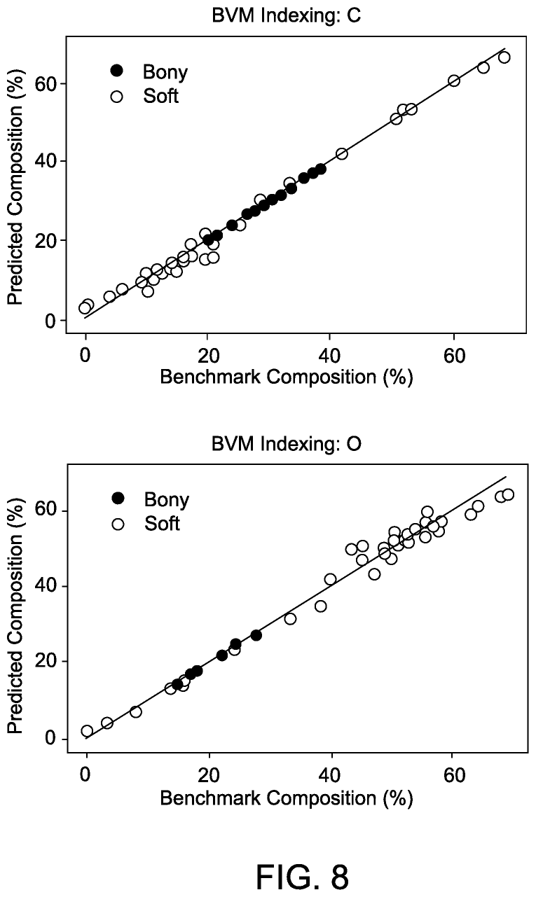

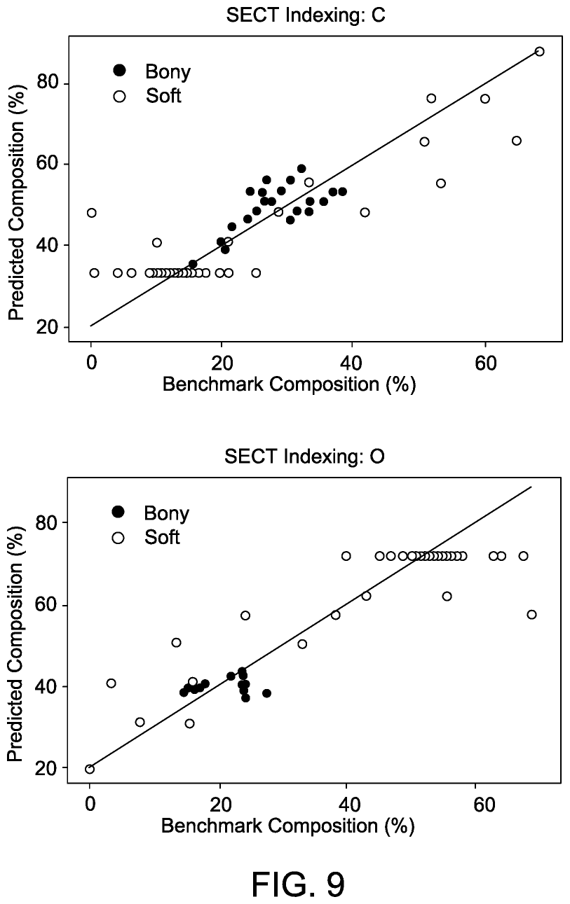

In reference to FIGS. 8 and 9, an elemental mass fraction of C and O estimated by BVM and SECT material indexing methods vs. benchmark compositions, where the straight line indicates a perfect correspondence.

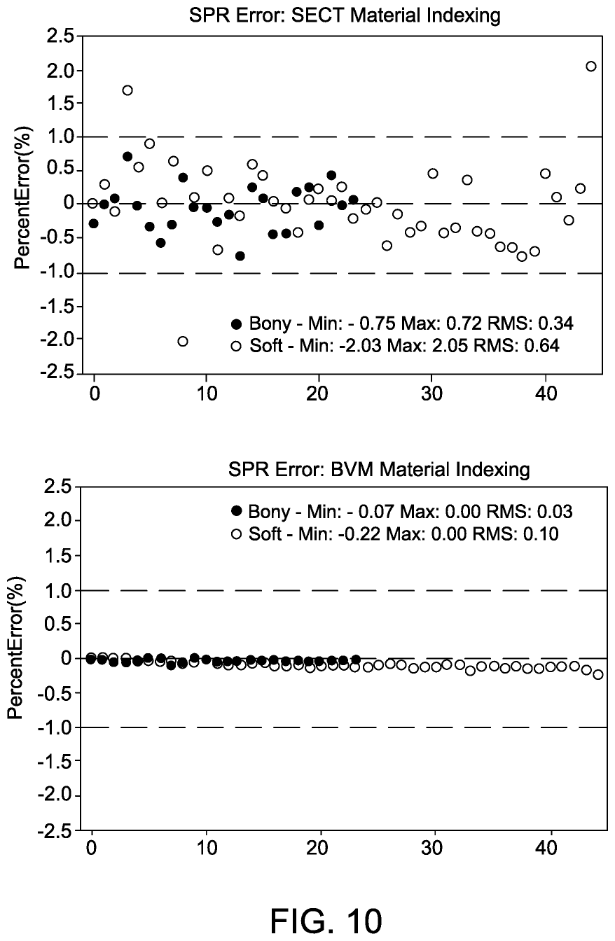

In reference to FIGS. 10-13, the Stopping Power Ratio (SPR) may be calculated using Bethe-Bloch Equation using predicted composition. Integrated depth dose curves for tissues with largest R80 difference between BVM and SECT material indexing. R80 Difference may be between 1-5 mm.

The proposed BVM material indexing method predicted SPR more accurately than standard SECT material indexing with SPR RMSE of 0.1% and 0.6%, respectively. The BVM material indexing predicted R80 more accurately than SECT material indexing with R80 RMSEs of 0.2 mm and 1.4 mm, respectively. The systems and methods described herein may be applied to determine MC lateral dose profiles in both the core region and the halo regions.

### Example 3

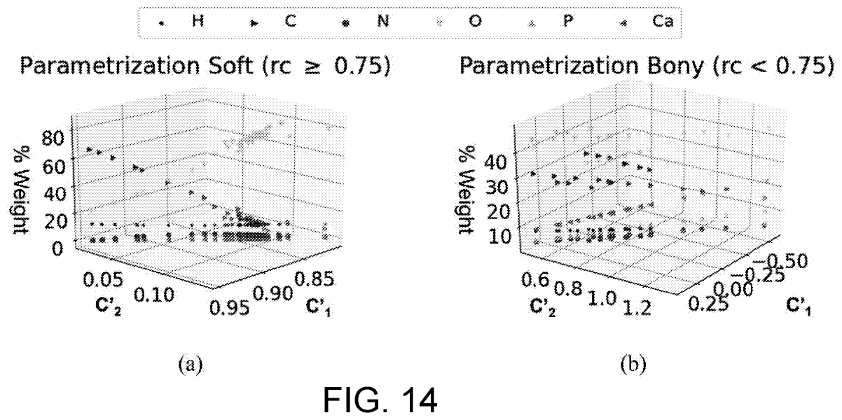

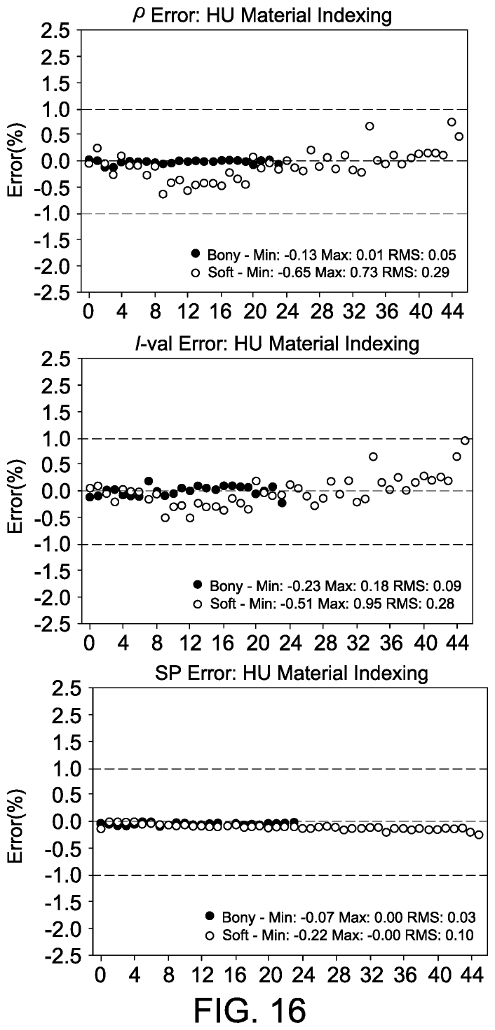

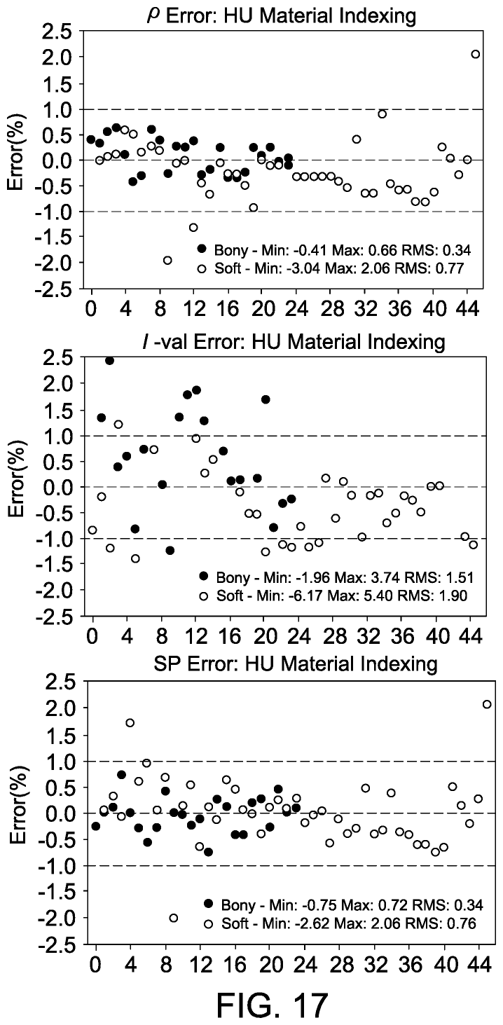

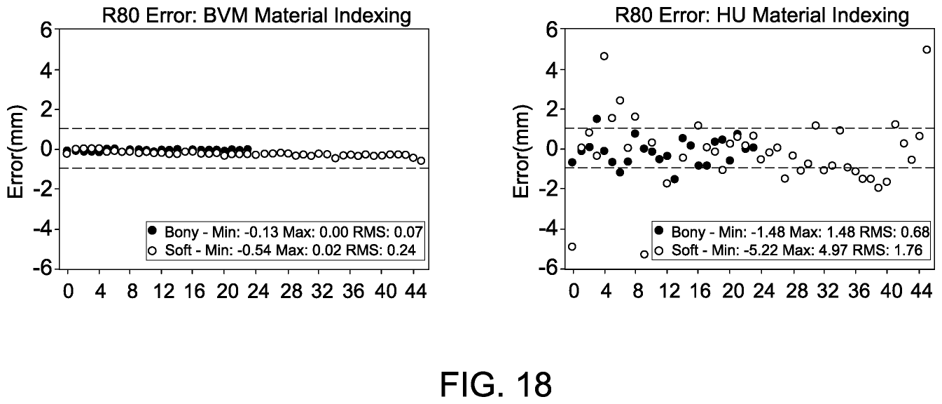

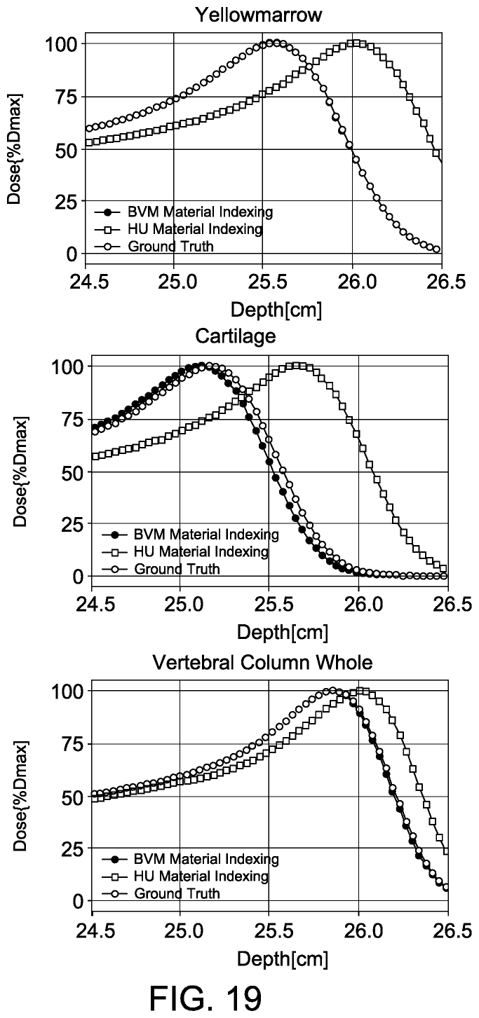

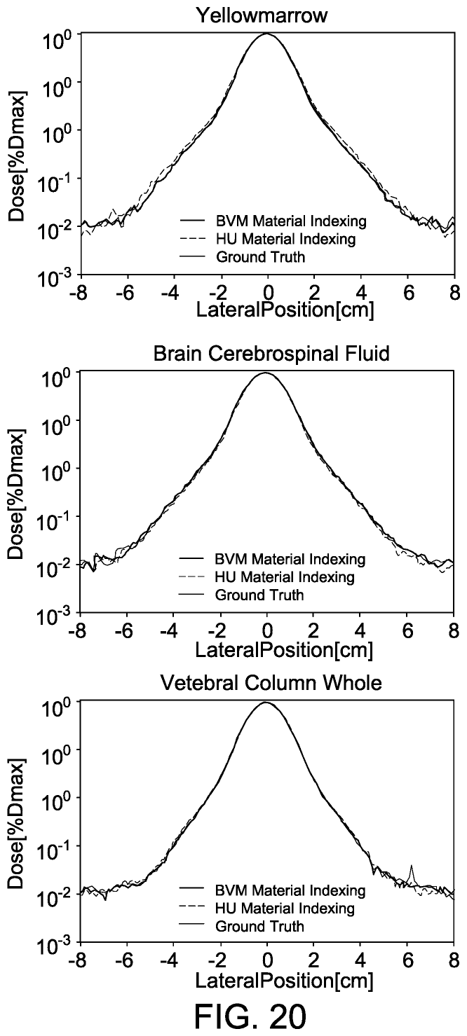

In reference to FIGS. 14-20, a third example, example 3, regarding a Derivation of Tissue Properties from Basis-Vector Model Weights for Dual-Energy CT-Based Monte Carlo Proton Beam Dose Calculations is provided.

Previous studies demonstrate that a linear basis-vector model (BVM) accurately predicts proton stopping-power ratio (SPR) maps in simulated and experimental data. While SPR is sufficient for pencil-beam dose calculations, Monte Carlo (MC) simulation requires the atomic composition and density of each medium to compute multiple elastic, inelastic, and nuclear scattering cross-sections. Embodiments described herein include methods for predicting atomic composition and mass density from the two independent BVM weights derived from dual-energy CT imaging.

Dose-calculation algorithms based on transport equation solutions support accurate dose computation in complex inhomogeneous geometries, provided that problem geometry, radiation sources, and all relevant cross-section data are accurately characterized. However, unlike pencil-beam dose calculations in which values of only one or two derived quantities (SPR, scattering power, and/-or mass density) need to be assigned to each material, Monte Carlo algorithms require a complete characterization of atomic composition and density of each medium so that the extensive library of cross sections needed to simulate elastic, inelastic, and nuclear scattering processes as a function of proton energy as well as cross-sections needed to support transport of all secondary particles (electrons, neutrons, gamma rays, and alpha particles). Given the limited one—to two-parameter per-voxel information extracted from SECT and DECT scans, a set of assumptions must be made for conversion of these data to mass-density and elemental fractional composition, to ensure that the problem is well-posed. The technique for such material characterization proposed in recent literature can be generally classified as segmentation, decomposition, and parametrization methods. In segmentation methods, the voxel wise reconstructed information, such as ρₑ or Z_{eff}, is used to find the closest material from a predefined set of reference tissues; then the known elemental information of the most similar tissue is assigned to the voxel. An alternative is to use decomposition methods which assume that a material can be broken down into a combination of fundamental components of known composition (such as lipid, protein, and water). CT measurements are then used to determine the fraction of each component, and from which the elemental composition of the investigated tissue can be inferred. In some embodiments, an extension of the decomposition technique worth mentioning is the principal component analysis (PCA) material characterization method, which uses PCA to represent human tissue information in an orthogonal basis and can further be implemented on multi-energy CT data.

The remaining material characterization techniques in the literature can then be classified as parametrization-based methods. These techniques parametrize the fractional information of each element as a function of one or more radiological quantities by performing fits on a database of human tissues of known composition. In doing so, they allow for a continuous estimation of elemental composition from DECT data. In embodiments described herein, a parametrization method for continuously predicting atomic composition and mass density from the two independent BVM weights derived from dual-energy CT imaging and compare it to a standard SECT and alternative DECT parametrization methods. Our proposed method exploits the full information derived from JSIR-BVM (in contrast to segmentation methods) to finally be able to be used not only for analytical but also future model-based proton-therapy dose calculations.

Our method, called BVM material indexing, uses multiple linear regression on the BVM weights and their quotient to predict the percent by weight concentration of elements for Z=1:20 and mass density of 69 representative tissue-compositions derived from the literature. The predicted compositions and densities were imported to the TOPAS MC codes and used to simulate a single 200 MeV proton beam delivered to uniform cylinder phantoms composed of the 69 tissues. MC dose distributions based on the BVM and a standard single-energy CT (SECT) material indexing approaches were compared to those derived from ground-truth tissue atomic compositions. The SPR, range (RBP), and depth of 80% of maximum dose (R80) were utilized to quantify dose-estimation errors.

The following sections present BVM material indexing, a method for directly estimating the atomic composition and density of an unknown tissue from the highly accurate weights (c₁ and c₂) derived from the basis vector model. The accuracy of the radiological quantities (ρₑ, I, and SPR) and Monte Carlo dose distributions predicted by our BVM material indexing method for 70 tissues (46 soft and 24 bony) of known elemental composition (see Supplementary Material S2) were compared to those of a standard SECT material-indexing approach. Our implementation of the BVM material indexing model was built and evaluated in Python 3.8.3 and Monte Carlo simulations were run on TOPAS 3.5 Toolkit, a user-friendly extension of Geant4.

### Basis Vector Model

BVM models the linear attenuation coefficients of an arbitrary material at the typical energies for a CT scan as the linear combinations of two basis materials

$$μ ( x , E ) = {c}_{1} ( x ) {μ}_{1} ( E ) + {c}_{2} ( x ) {μ}_{2} ( E ) , \qquad ( 1 )$$

Where μᵢ are the linear attenuation coefficients of basis materials i=1:2 for photon energy E and cᵢ(x) denotes the basis material weight at image voxel location x. For this theoretical study, basis materials Polystyrene and 23% aqueous solution of CaCl₂) were selected as they have shown to model linear attenuation coefficient of materials with atomic numbers of 2 to 20 with a 1-2% accuracy in the 20 keV to 1 MeV energy range. Other potential basis materials may be selected. Following the basis vector model, the predicted maps of electron density (ρₑ(x)) and mean excitation energy (I(x)) can be calculated by the linear relationships

$${ρ}_{e} ( x ) = {c}_{1} ( x ) {ρ}_{e , 1} + {c}_{2} ( x ) {ρ}_{e , 2} , \qquad ( 2 )$$

$$I ( x ) = exp ( a \cdot {r}_{c} ( x ) + b ) ,$$

Where ρ_{e,i} denotes the electron density of material i and where a and b in (2) are derived by fitting I-values for tissues with known atomic composition to the electron density-weighted basis-component fraction

$${\hat{r}}_{c} ( x ) = \frac{{\hat{c}}_{1} ( x ) {ρ}_{e , 1}}{{\hat{c}}_{1} ( x ) {ρ}_{e , 1} + {\hat{c}}_{2} ( x ) {ρ}_{e , 2}} , \qquad ( 3 )$$

which has previously been used as a surrogate for tissue composition in I-value calculations and has proved to be effective in estimating I-values with errors of 2.36%.

The values of c₁, c₂, and r_{c} for an arbitrary material can be derived by applying image and sinogram decomposition approaches on dual-energy CT data or directly through iterative reconstruction methods. To test the BVM material indexing method, theoretical values of c₁, c₂, and r_{c} for 70 reference tissues were derived by using the minimum least-square approach outlined in previous studies.

### BVM Material Indexing

The proposed indexing method, hereafter known as BVM material indexing, estimates an elemental composition of an arbitrary tissue, y, by a multiple linear model on the reconstructed JSIR-BVM basis-vector model weights, c_{{1,2},y} and their corresponding weighted component ratio (r_{c}). To allow for the incorporation of low-density lung into the final parametrization, BVM material indexing uses a scaled version of the basis-material weights (c′_{{1,2},y}) and weighted component ratio (r′_{{c,y}}) in the final elemental parametrization of tissue y. The parameters c′_{{1,2},y} and r′_{{c,y}} can be defined as

$${c}_{\{ 1 , 2 \} , y}^{′} = {c}_{\{ 1 , 2 \} , y} \cdot \frac{{ρ}_{e , w}}{{ρ}_{e , y}} and {r}_{\{ c , y \}}^{′} = \frac{{c}_{1 , y}^{′} {ρ}_{e , 1}}{{c}_{1 , y}^{′} {ρ}_{e , 1} + {c}_{2 , y}^{′} {ρ}_{e , 2}} ,$$

where ρ_{e,y} is the predicted electron density of tissue y and ρ_{e,1} and ρ_{e,2} are the electron densities of basis material c_{1,y} and c_{2,y}, respectively. The linear relationship between the scaled JSIR-BVM reconstructed basis vector model weights and weighted component ratio and a material's mass fraction for each element k can then be expressed as

$${w}_{k , x} = {α}_{1 , k} {c}_{1 , y}^{′} + {α}_{2 , k} {c}_{2 , y}^{′} + {α}_{3 , k} {r}_{c , y}^{′} + {α}_{4 , k} , \qquad ( 4 )$$

The k denotes one of the six main elements included in this study (hydrogen, carbon, nitrogen, oxygen, phosphorus, and calcium), and c′_{1,y}, c′_{2,y}, and r′_{c,y} denote the scaled estimated basis vector model weights and weighted component ratio, respectively. The final α parameters can be obtained by performing a multiple linear regression fitting between theoretical mass fractions (wₚ) of elements p and benchmark c′₁, c′₂, and r′_{c} for the 70 reference tissues with material compositions normalized to the six major elements used in this work. Thyroid was not included in our comparison as it has previously shown high BVM modelling errors due to its iodine content (Z=53). Because of compositional differences, most apparently on phosphorus and calcium content, two sets of linear regression models (illustrated in FIG. 1) were trained, one for the bony-(r′_{c}<0.75) and one for the soft-tissue r′_{c} range (r′_{c}≥0.75). The predicted elemental mass fractions of the 6 most common elements in human tissues, hydrogen, carbon, nitrogen, oxygen, phosphorus, and calcium, for each tissue were then normalized to add up to 1, and mass densities were finally calculated by applying the atomic mixture rule to the six elemental weights predicted through the BVM model and the electron densities derived with equation (2).

### Performance Evaluation and Monte Carlo Range Studies

The accuracy of HU and BVM material-indexing methods was evaluated by means of: (1) the discrepancy between radiological quantities derived from benchmark and predicted elemental compositions, and (2) differences between proton-beam Monte Carlo-calculated dose distributions based on predicted tissue compositions (by one of the two analyzed methods) and simulations based on benchmark elemental compositions and densities.

The radiological quantities in comparison (1) included electron density, I-value, and stopping power. The mass density (ρₘ) and elemental mass fraction, wₚ was first evaluated for each indexing method. Next, the atomic mixture and Bragg additivity rules were used to estimate ρₑ and I. Finally, the stopping power of the material at energy Eₚ was calculated using a simplified version of the Bethe-Bloch equation without density and shell corrections which are negligible for the clinically relevant energy range. The resulting values of ρₘ, I, and SP were finally compared to their corresponding values computed from the ground-truth densities and elemental composition. The three analyzed methods were also compared in terms of the percentage point difference between predicted and benchmark elemental composition for the six main elements considered in this study.

In some embodiments, the BVM material indexing method may be evaluated for overfitting, such as a 5-fold cross validation for each of the six studied elemental parametrizations. For each elemental parametrization, the bony and soft tissue sets were split into five folds; in each iteration one of the five disjoint sets were used for testing while the remaining data was used for training until all five folds were used. An average of the percentage point error for all five iterations was reported.

To analyze the performance of different material parametrization methods in MC simulations, a ground truth may be imported, HU, and BVM material-indexing densities and mass fractional atomic compositions for the 70 evaluated tissues into TOPAS Version 3.5 which shares the same physics models and interaction cross-sections as Geant4 10.06.p01. All the described TOPAS simulations were run using the default physics lists provided by Geant4: g4em-standard_opt4, g4h-phy_QGSP_-BIC_HP, g4decay, g4ion-binarycascade, g4h-elastic_HP, and g4stopping. The influence of different material indexing methods in Monte Carlo dose calculations was assessed by evaluating the absorbed dose from proton pencil beam in phantoms composed of materials defined by one of the three different material indexing techniques. TOPAS was used to compute the integral depth dose (IDD) of the beam, which was quantified by evaluating its range at 80% of its maximum dose (R80). In our simulations, a 200 MeV monoenergetic cylindrically symmetric parallel proton beamlet consisting of 2×10⁵ particles in a Gaussian distribution with a standard deviation of 4 mm was delivered to a solid cylinder phantom of 50 cm radius and 30 cm in length composed of one of the 70 analyzed tissues.

In the case of inflated lung, the monoenergetic beam was simulated at 75 MeV to ensure that the range of the IDD was within the total length of the cylinder. The dose to medium for all simulations were scored along the beam axis in 800 disk-shaped bins, each 0.38 mm thick. The standard deviations scored sum ranged from 0.2% to 0.5% in the [0, R80] depth range. To calculate R80, the resultant IDD curve was fit to a Bortfeld fit and then the depth of the distal 80% of the maximum dose was measured. The spatial spread of the proton beam due to multiple coulomb scattering and secondary neutral particle (gamma rays and neutrons) formation was assessed in additional simulations by scoring dose in lateral profiles at two representative depths: the Bragg peak at R80 depth and the plateau region at 50% of the R80 depth. The same monoenergetic proton beam used in our integrated depth dose simulations was used. In this case the proton beam was delivered to a solid cylinder of 10.025 cm in radius and 40 cm in length, and the lateral dose to medium was scored perpendicular to the beam axis in 401 bins of 0.5 mm height and 5 mm radial thickness. The z position of the scorer was dynamically changed based on the R80 position calculated for each tissue and material indexing method. Each scored lateral dose was then fit to a cubic spline and the spread of the beam was quantified by calculating the full-width half maximum of the lateral dose at 80% of the maximum dose (FWHM80). The final overall performance of both material indexing methods was assessed by evaluating the difference in mm between the R80 and FWHM80 calculated in the Monte Carlo simulations with materials defined by BVM and HU material libraries and those calculated in simulations with materials defined by benchmark compositions.

Root-mean-square (RMS)/Max error in estimated SPR and RBP were 0.6/2.1% and 1.3/5.2 mm for SECT and 0.1/0.3% and 0.3/0.6 mm for BVM material-indexing schemes. Similarly, RMS/Max R80 errors for bony (soft) tissues for the SECT and BVM approaches were 0.7/1.5 mm (1.6/5.3 mm) and 0.1/0.3 mm (0.3/0.7 mm), respectively.

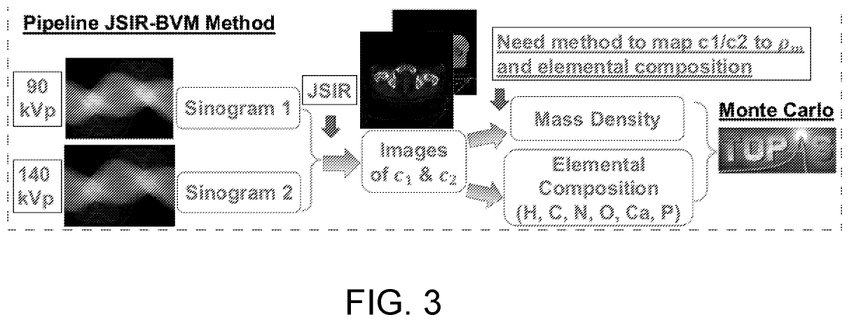

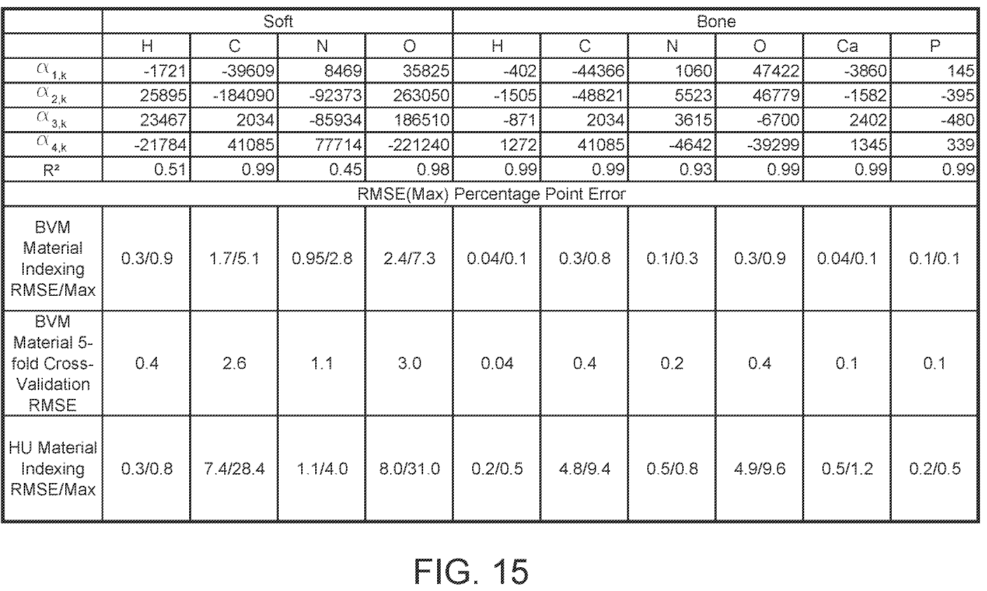

FIG. 15 shows the final BVM material indexing fit applied on benchmark fractional elemental compositions and the overall accuracy of HU and BVM material indexing methods in predicting elemental weight fractions for soft and bony tissues. As can be observed, BVM estimated the elemental fractional composition of the analyzed bony and soft tissues with higher accuracy than the conventional HU method. The most significant differences can be observed in the estimation of (carbon/oxygen) content for which HU and BVM material-indexing had RMSEs of 7.4/8.0% and 1.7/2.4% for soft tissue and 4.8/4.9% and 0.3/0.3% for bony tissue. Our 5-fold cross-validation RMSE was comparable to our overall RMSE, confirming the generalizability of our method when applied to the given reference tissue library. To further illustrate differences in performance between the two analyzed methods, scatter plots comparing estimated and benchmark oxygen and carbon elemental fractions for the 70 investigated tissues are provided in Supplementary Material S1. As can be observed, the BVM parametrization results very closely follow the black line which indicates perfect correspondence between predicted and benchmark values. FIG. 3 shows the percent error difference between benchmark and predicted ρₘ, I-value, and SP for the two analyzed parametrization methods. The proposed BVM parametrization method showed better correspondence to soft/bony tissue benchmark values with RMS ρₘ, I-value, and SP errors of 0.29/0.05%, 0.28/0.09%, and 0.10/0.03% than the conventional HU material indexing method (0.77/0.34%, 1.90/1.51%, and 0.76/0.34%).

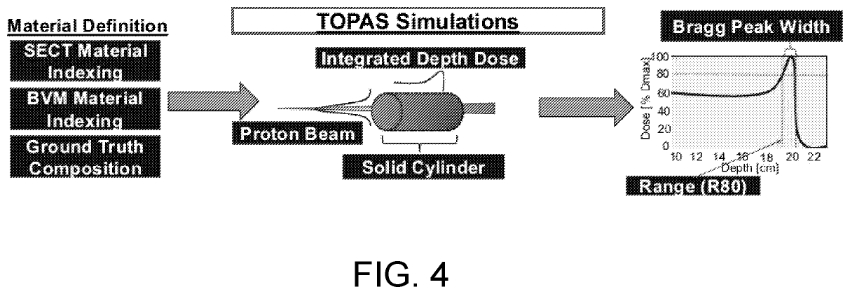

Our Monte Carlo performance analysis is summarized in FIG. 4. As can be observed, the elemental compositions estimated by the proposed BVM material indexing method support more accurate R80 localization than the HU material indexing method with soft/bony RMS errors of 0.24/0.07 mm for BVM and 1.63/0.68 mm for HU parametrization method. The reference soft tissues with the three highest R80 estimation errors for HU were brain cerebrospinal fluid, cartilage, and inflated lung; on the other hand, for BVM, the soft tissues with the highest R80 error were cartilage, eye lens, and connective tissue.

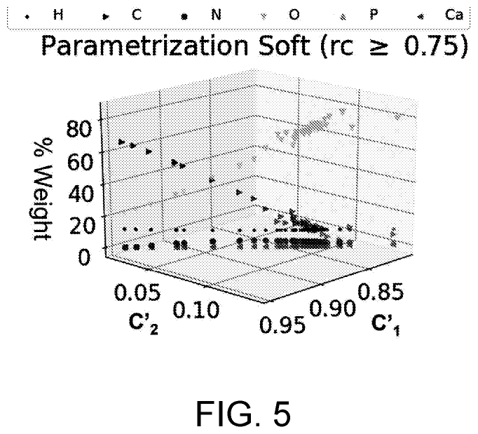

In terms of lateral dose profiles, no significant differences between models in predicting lateral beam spread of the beam were observed. The overall soft/bony RMS errors of the FWHM80 at Bragg peak for BVM and HU were 0.08/0.05 mm and 0.18/0.11 mm, respectively. FIG. 4 and FIG. 5 further illustrate the discrepancies in more detail by showing IDD and lateral dose profiles for tissues with the largest R80 and FWHM80 discrepancies. The observed differences in R80 between ground truth (red line) and HU material indexing (blue line) ranged from 1 to 5 mm.

Our results show that fully exploiting the two-parameter BVM space for material indexing dramatically improves TOPAS MC dose-calculation accuracy (by factors of 4 to 7 in RMS) compared to the standard SECT single-parameter indexing process.

### Model Enhancement

In embodiments described herein may include implementing the proposed BVM material indexing method is to test it on basis-vector basis images reconstructed with our iterative JSIR-BVM method from raw sinogram data. In some embodiments, the method and systems described herein may include determining the optimal number of TOPAS materials to be defined from reconstructed images and incorporating them into the TOPAS simulation code. In some embodiments, methods may include implementing a k-means clustering on pixel-wise reconstructed elemental compositions and developing an in-house TOPAS extension. In some embodiments, testing the BVM material indexing method may be evaluated in simulation scenarios with heterogeneous tissue compositions.

### Implementation of BVM-Material Indexing in MECT and Deep Learning

The BVM material indexing method is a two-parameter based calibration method to determine six material composition parameters. Deep learning technique may also be used to directly learn the optimal α weights and to map directly from reconstructed c′₁, c′₂, and r′_{c} maps to estimated fractional elemental composition. By shifting from a pixel-wise to an image-wise analysis using deep learning, in some embodiments, methods may include utilizing geometric priors to enforce geometrical correlations among voxels including organ types and boundaries. Digital phantom images may be used to train a U-Net and VGG network architecture to predict elemental composition fractions from reconstructed single-, dual-, and quad-energy CT data. Segmentation tools along with the spectral and geometrical information from the DECT system, digital phantoms may be generated based on real DECT data. A network may be trained to map the basis vector model weights to the desired elemental fraction composition. Additionally, given that reconstructed BVM weights are robust to noise and beam hardening artifacts, our BVM material indexing network would have the potential to better focus on learning the mapping to fractional elemental composition than other DECT mapping methods that are susceptible to noise in low and high energy CT input images.

### Other Possible Applications of BVM Material Indexing

Embodiments described herein may include using a BVM material indexing method to derive the material properties (mass density and fractional elemental composition) of biological tissues from DECT data for accurate Monte Carlo based proton beam dose calculations. Besides being able to apply this method for direct proton beam dose verifications, the BVM material indexing method could also be used in treatment verification based on proton-induced secondary signals through nuclear reactions. This would be especially helpful in the Monte Carlo modelling of PET activity profiles where uncertainties in the derivation of carbon and oxygen elemental concentrations could lead to range uncertainty of up to 1 mm. Other possible applications of BVM material indexing are dose calculations in hadron therapy with carbon ions and future classification of healthy and cancerous tissue by taking advantage of the higher hydrogen concentration in tumors.

### Example 4

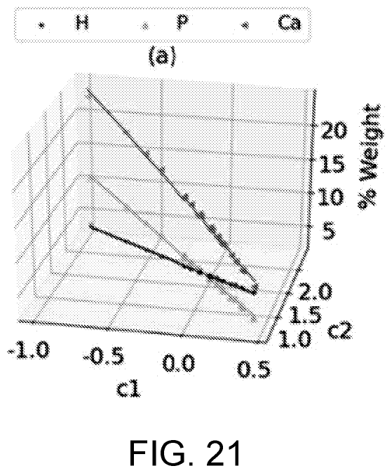

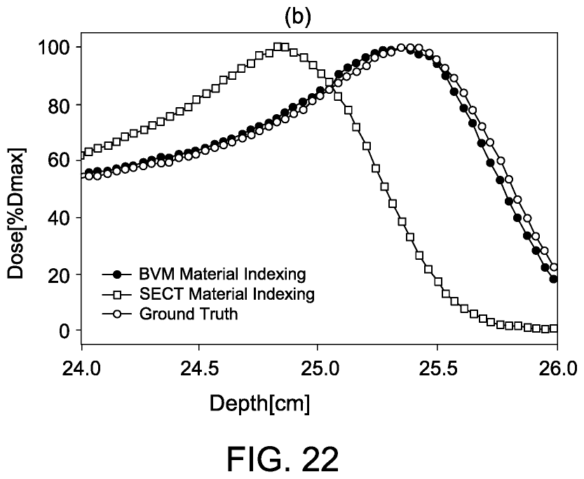

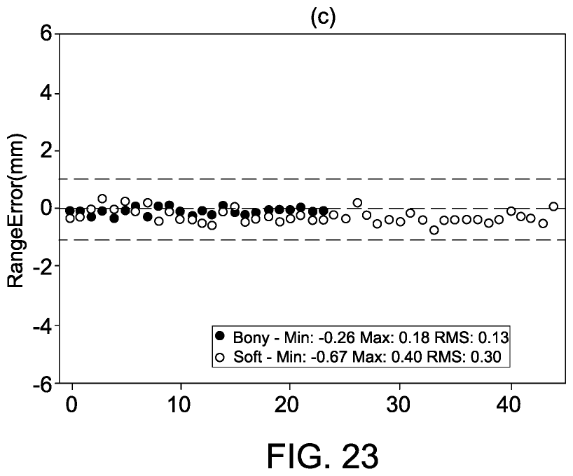

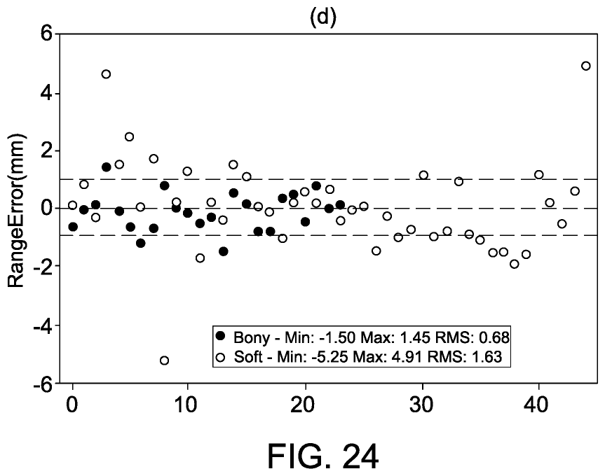

In reference to FIGS. 21-24, a fourth example regarding predicting elemental composition of bony and soft tissues predicted using BVM weights, is provided.

This study demonstrates for the first time that the elemental composition of bony and soft-tissues can be predicted from their BVM weights with an accuracy sufficient to model proton-beam depth-dose curves with an accuracy of ±0.3 mm via TOPAS MC simulations. This is important because (a) the linear separable BVM is the only model compatible with joint statistical iterative DECT reconstruction (JSIR) based on the polyenergetic forward projection model and (b) JSIR has been demonstrated to predict proton ranges with much better accuracy and lower uncertainties than the more common image based DECT material decomposition methods. BVM material indexing is the key link for integrating these two powerful methods: JSIR DECT material characterization and proton Monte Carlo dosimetry.

BVM models linear attenuation coefficients, μ(x, E)=cᵢ(x)μ₁(E)+c₂(x)μ₂(E), as linear combinations of two basis materials where c₁ and c₂ are the basis component weights for the polystyrene and CaCl₂) solution basis pair assumed by this work. The corresponding basis-weight ratio is r_{c}=(ρ_{e,i} cᵢ)/(ρ_{e,i} cᵢ+ρ_{e,2} c₂), where ρ_{e,i} denotes electron density of basis material i. BVM material indexing predicts the elemental mass fraction, wᵢ (for i=1:6 for 6 most common elements in human tissues) of an arbitrary tissue from its c₁, c₂, and r_{c}. Our study used the theoretical values of c₁, c₂, and r_{c} for 69 representative tissues derived from their benchmark atomic composition and CT scanning spectra as outlined in previous studies. Then, the mass fractions of the six main elements (H, C, N, O, Ca, and P) were fit to the theoretical c₁, c₂, and r_{c} values by two sets of linear regression models (wᵢ=α_{1,i}c₁+α_{2,i},c₂+α_{3,i},r_{c}+α_{4,i}) for bony tissue (r_{c}<0.75) and (wᵢ=β_{1,i}c₁+β_{2,i},c₂+β_{3,i},r_{c}+β_{4,i}) for soft tissue (r_{c}≥0.75). Mass fractions were then normalized to add up to 1. Mass density values were derived by applying the atomic mixture rule to ρₑ=ρ_{e,1}c₁+ρ_{e,2}c₂) and the predicted wᵢ. Figure (a) illustrates the high quality of the linear curve fits obtained. Methods include a BVM voxel indexing and a well-established SECT material-indexing method to the ground truth compositions in terms of predicted stopping power ratio and IDD curves predicted by TOPAS for each composition

For the soft tissue with the largest discrepancy, Figure (b) demonstrates that the MC-predicted IDD curve based on the BVM material indexing composition, closely tracks the ground truth IDD curve while the SECT method underestimates range by more than 5 mm. Figure (c) and (d) summarize the errors in the range of 80% (R80) of the maximum integrated depth dose. Overall, the R80 derived from our method showed better performance than those derived from a standard SECT material indexing method.

The systems and methods described herein may be implemented in any suitable computing device 800 and software implemented therein. FIG. 25 is a block diagram of an example computing device 800. In the example embodiment, the computing device 800 includes a user interface 804 that receives at least one input from a user. The user interface 804 may include a keyboard 806 that enables the user to input pertinent information. The user interface 804 may also include, for example, a pointing device, a mouse, a stylus, a touch sensitive panel (e.g., a touch pad and a touch screen), a gyroscope, an accelerometer, a position detector, and/or an audio input interface (e.g., including a microphone).

Moreover, in the example embodiment, computing device 800 includes a display interface 817 that presents information, such as input events and/or validation results, to the user. The display interface 817 may also include a display adapter 808 that is coupled to at least one display device 810. More specifically, in the example embodiment, the display device 810 may be a visual display device, such as a cathode ray tube (CRT), a liquid crystal display (LCD), a light-emitting diode (LED) display, and/or an “electronic ink” display. Alternatively, the display interface 817 may include an audio output device (e.g., an audio adapter and/or a speaker) and/or a printer.

The computing device 800 also includes a processor 814 and a memory device 818. The processor 814 is coupled to the user interface 804, the display interface 817, and the memory device 818 via a system bus 820. In the example embodiment, the processor 814 communicates with the user, such as by prompting the user via the display interface 817 and/or by receiving user inputs via the user interface 804. The term “processor” refers generally to any programmable system including systems and microcontrollers, reduced instruction set computers (RISC), complex instruction set computers (CISC), application specific integrated circuits (ASIC), programmable logic circuits (PLC), and any other circuit or processor capable of executing the functions described herein. The above examples are example only, and thus are not intended to limit in any way the definition and/or meaning of the term “processor.”

In the example embodiment, the memory device 818 includes one or more devices that enable information, such as executable instructions and/or other data, to be stored and retrieved. Moreover, the memory device 818 includes one or more computer readable media, such as, without limitation, dynamic random-access memory (DRAM), static random-access memory (SRAM), a solid-state disk, and/or a hard disk. In the example embodiment, the memory device 818 stores, without limitation, application source code, application object code, configuration data, additional input events, application states, assertion statements, validation results, and/or any other type of data. The computing device 800, in the example embodiment, may also include a communication interface 830 that is coupled to the processor 814 via the system bus 820. Moreover, the communication interface 830 is communicatively coupled to data acquisition devices.

In the example embodiment, the processor 814 may be programmed by encoding an operation using one or more executable instructions and providing the executable instructions in the memory device 818. In the example embodiment, the processor 814 is programmed to select a plurality of measurements that are received from data acquisition devices.

In operation, a computer executes computer-executable instructions embodied in one or more computer-executable components stored on one or more computer-readable media to implement aspects of the invention described and/or illustrated herein. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

Example embodiments of systems and methods of adaptive radiotherapy are described above in detail. The systems and methods are not limited to the specific embodiments described herein but, rather, components of the systems and/or operations of the methods may be utilized independently and separately from other components and/or operations described herein. Further, the described components and/or operations may also be defined in, or used in combination with, other systems, methods, and/or devices, and are not limited to practice with only the systems described herein.

Although specific features of various embodiments of the invention may be shown in some drawings and not in others, this is for convenience only. In accordance with the principles of the invention, any feature of a drawing may be referenced and/or claimed in combination with any feature of any other drawing.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A computer-implemented method of predicting planning parameters for Monte Carlo proton radiation planning on a subject, comprising:
receiving training data of tissues, wherein for each tissue, the training data include elemental compositions of a plurality of elements in the tissue, a mass density of the tissue, and basis material weights when a linear attenuation coefficient of the tissue is represented as a weighted sum of linear attenuation coefficients of basis materials;
executing a linear regression model, wherein an elemental composition includes a mass fraction of one of the plurality of elements in a tissue, and the model includes that the mass fraction is a linear function of the base material weights; and
training the model by:
fitting the training data with the model; and
adjust model parameters of the model during the fitting,
wherein the trained model is configured to predict planning parameters required in a Monte Carlo proton radiation planning system in generating a proton radiation plan of a subject, wherein the planning parameters include elemental compositions and mass densities of tissues in a treatment region of the subject.

2. The method of claim 1, wherein the model includes that the mass fraction is a linear function of a weighted component ratio between the basis material weights.

3. The method of claim 2, wherein the weighted component ratio is derived based on the basis material weights and electron densities of the basis materials.

4. The method of claim 2, wherein the model includes a first submodel of a soft tissue and a second submodel of a bony tissue, and fitting the training data further comprises:
bracketing materials into the soft tissue and the bony tissue based on the weighted component ratio; and
applying the first submodel or the second submodel on the training data based on the bracketing.

5. The method of claim 1, wherein receiving training data of tissues further comprises:
normalizing the basis material weights to account for a tissue having a low mass density into the model by scaling the basis material weights with a factor.

6. The method of claim 5, wherein normalizing the basis material weights further comprises scaling the basis material weights with the factor as a ratio between electron densities of the basis materials.

7. The method of claim 5, wherein the model includes that the mass fraction is a linear function of the normalized basis material weights and a weighted component ratio between the normalized basis material weights.

8. The method of claim 1, wherein training the model further comprises:
for each tissue,
estimating the mass density of the tissue based on electron densities of the basis materials and the basis material weights;
comparing the estimated mass density with the mass density of the tissue in the training data; and
adjusting the model based on the comparison.

9. A computer-implemented method of predicting planning parameters for Monte Carlo proton radiation planning on a subject, comprising:
receiving raw data of a subject acquired using a computed tomography system;
deriving basis material weights at each image voxel location based on the raw data, wherein the basis material weights are weights in expressing a linear attenuation coefficient of a tissue at the image voxel location as a weighted sum of linear attenuation coefficients of basis materials;
execute a linear regression model;
estimating planning parameters including elemental compositions of a plurality of elements and/or a mass density of the tissue at the image voxel location based on the basis material weights using the model; and
outputting the planning parameters, wherein the planning parameters are required in a Monte Carlo proton radiation planning system in generating a proton radiation plan of the subject.

10. The method of claim 9, wherein each elemental composition includes a mass fraction of one of the plurality of elements, and the model includes that the mass fraction is a linear function of the basis material weights.

11. The method of claim 10, wherein the model includes that the mass fraction is a linear function of a weighted component ratio.

12. The method of claim 11, wherein the model includes a first submodel of a soft tissue and a second submodel of a bony tissue, and estimating planning parameters further comprises:
bracketing materials into the soft tissue and the bony tissue based on the weighted component ratio; and
estimating the planning parameters by applying the first submodel or the second submodel based on the bracketing.

13. The method of claim 9, wherein deriving basis material weights further comprises:
normalizing the basis material weights to account for a tissue having a low mass density in the model by scaling the basis material weights with a factor.

14. The method of claim 13, wherein normalizing the basis material weights further comprises scaling the basis material weights with the factor as a ratio between electron density of the basis materials.

15. The method of claim 13, wherein each elemental composition includes a mass fraction of one of the plurality of elements, and the model includes that the mass fraction is a linear function of the normalized basis material weights and a weighted component ratio between the normalized basis material weights.

16. The method of claim 9, wherein estimating planning parameters further comprises:
estimating the mass density based on electron densities of the basis materials and the basis material weights.

17. A computer-implemented method of predicting planning parameters for Monte Carlo proton radiation planning, comprising:
receiving raw data of a subject acquired using a computed tomography system;
deriving basis material weights of a plurality of basis materials at an image voxel location based on the raw data;
execute a machine learning model;
estimating planning parameters including elemental compositions of a plurality of elements and/or a mass density of tissues in the image voxel location using the model based on the basis material weights; and
outputting the planning parameters, wherein the planning parameters are required in a Monte Carlo proton radiation planning system in generating a proton radiation plan of the subject.

18. The method of claim 17, wherein an elemental composition includes a mass fraction of one of the plurality of elements, and the model includes that the mass fraction is a function of the basis material weights and a weighted component ratio.

19. The method of claim 18, wherein the model includes a first submodel of a soft tissue and a second submodel of a bony tissue, and estimating planning parameters further comprises:
bracketing materials into the soft tissue and the bony tissue based on the weighted component ratio; and
estimating the planning parameters by applying the first submodel or the second submodel based on the bracketing.

20. The method of claim 18, wherein deriving basis material weights further comprises:
normalizing the basis material weights to account for a tissue having a low mass density in the model by scaling the basis material weights with a factor,
wherein the model includes that the mass fraction is a linear function of normalized basis material weights and a weighted component ratio between the normalized basis material weights.
